(19) United States
(12) Patent Application Publication
(10) Pub. No.: US 2005/0202414 A1
(43) Pub. Date: Sep. 15, 2005

(54) Apparatus and methods for detecting a microbe in a sample

(75) Inventors: Xi Yu Jia, Irvine, CA (US); Martina M. Berger, Newport Beach, CA (US); Jeremiah G. Tilles, Irvine, CA (US)

Correspondence Address: Robert D. Buyan;Stout, Uxa, Buyan & Mullins, LLP, Suite 300, 4 Venture, Irvine, CA, 92618, US

(73) Assignee: The Regents of the University of California, Oakland, CA (US)

(21) Appl. No.: 10/295,787

(22) Filed: Nov. 15, 2002

Related U.S. Application Data
(60) Provisional application No. 60/335,539, filed on Nov. 15, 2001.

(51) Int. Cl. C12Q 1/70; C12Q 1/68; C12M 1/34

(52) U.S. Cl. 435/5; 435/287.2; 435/6

(57) ABSTRACT

Apparatus for detecting one or more microbes in a sample includes a substrate having a plurality of microbe identification sites with nucleic acid probes disposed thereon, each nucleic acid probe having a nucleotide sequences that is complementary to nucleotide sequences of nucleic acids of one or more microbes. Nucleotide sequences for nucleic acid probes and the primers used to generate the probes are disclosed. Methods of detecting a microbe in a sample using nucleic acid probes are also disclosed.

### CROSS-REFERENCE TO RELATED APPLICATIONS

This application claims the benefit of U.S. Provisional Application No. 60/335,539, filed Nov. 15, 2001, the entire contents of which are hereby incorporated by reference.

### FIELD OF THE INVENTION

The present invention relates to apparatus and methods for detecting the presence of a microbe in a sample. More particularly, the invention relates to substrates, such as microarrays, having nucleic acid probes that hybridize to nucleic acids of microbes in a sample, the sequences of the nucleic acid probes, methods of detecting one or more microbes in a sample using nucleic acid probes, and nucleic acid primers for a polymerase chain reaction (PCR) of a microbe's nucleic acids.

### BACKGROUND

Of the millions of people that die each year, approximately thirty percent of the deaths are due to infectious diseases. Among infectious diseases, ten diseases that likely result in death include acute lower respiratory infections, diarrhoeal diseases, tuberculosis, malaria, hepatitis B, HIV/Aids, measles, neonatal tetanus, whooping cough (pertussis), and intestinal worm diseases. Roughly twenty-five percent of the deaths may be attributed to acute lower respiratory infections.

Many respiratory diseases may be caused by viral pathogens, which can be classified as double stranded DNA (dsDNA) viruses; double stranded RNA (dsRNA) viruses; retroid viruses, single stranded DNA (ssDNA) viruses; single stranded RNA (ssRNA) viruses, which may comprise either a plus strand (sense strand) or a minus strand (antisense strand); mononegavirales; and delta virus. Important human dsDNA viruses include adenoviridae, mastadenovirus, human adenovirus A (subtypes 12, 18, 31), human adenovirus B (subtypes 3, 7, 11, 14, 16, 21, 34, 35, 50); human adenovirus C (subtypes 1, 2, 5, 6, 13); human adenovirus D (subtypes 8, 9, 10, 13, 15, 17, 19, 19a, 19p, 20, 22, 23, 24, 25, 26, 27, 28, 29, 30, 32, 33, 36, 37, 38, 39, 42, 43, 44, 45, 46, 47, 48); human adenovirus E (subtype 4); human adenovirus F (subtypes 40, 41); herpesviridae, including alphaherpesvirinae (simplex virus: human herpes type 1, 2, 7; and varicellovirus: human herpes type 3), betaherpesvirinae (cytomegalovirus: human herpes type 5; and roseolovirus: human herpes type 6, 6A, 6B), gammaherpes virinae (lymphocryptovirus: human herpes 4 (Epstan Bar virus); and rhadinovirus: Kaposi's sarcoma associated herpesvirus—human herpes virus 8); papilomaviridae, including papilomavirus (human papilomaviruses in which there are more than 84 different types); polyomaviridae, including polyomavirus (JC virus, BK virus (AS and BS strains)); and poxviridae, including orthopoxvirus (vaccinia and small pox). Important human dsRNA viruses include reoviridae and orthoreovirus-rotaviruses. Important human retroids include hepadnaviridate (human hepatitis B virus); retroviridae; deltavirus (HTLV types 1 and 2); lentivirus (HIV1, HIV2, and HIV3); mammalia-type C retrovirus; spumavirus-spumaretrovirus; and type D retrovirus. Important human ssDNA viruses include parvoviridae erythrovirus (types B19 and V9) and adeno-associated virus (types 1-6). Important human ssRNA (−) viruses include arenaviridae-Lassa, lymphocytic choriomeningitis virus, bunyaviridae (of which there are at least twenty five different types); and hantavirus. Important human mononegavirales include borna disease; filoviridae, including Ebola virus and Marburg virus; paramyxovirnae, including Hendra virus, measles virus, human parainfluenza viruses 1, 2, 3, and 4, mumps, and respiratory syncytial virus; rhabdoviridae, including rabies; and orthomyxoviridae, including influenza A, B, and C. Important human ssRNA (+) viruses include astroviridae (human astrovirus types 1-7); caliciviridae (human calicivirus, and norwalkvirus), faviridae (which include over fifty viruses, including Dengue virus, Japanese encephalitis, St. Louis encephalitis, West Nile virus, human hepatitis C virus, and others); coronaviridae; picornaviridae, including aphthovirus, cardiovirus, enteroviruses, hepatovirus, rhinoviruses; and togaviridae, including rubella virus, alphavirus VEEV and WEEV, and more than fifteen other viruses. An important deltavirus includes the human hepatitis D virus.

Current testing procedures and devices for infectious diseases, such as radioimmunoassays and enzyme-linked immunosorbent assays (ELISAs), are difficult to implement, time consuming, expensive, and/or outdated. In addition, current procedures typically rely on the use of agents that recognize and bind to membrane bound proteins or carbohydrates of the pathogen. The shortcomings of conventional procedures may be due to the large number of different pathogens, the large numbers of different assays. In other words, there are too many choices, and not one choice that can assay for multiple pathogens. Thus, there is a need for a device that is compact, sensitive, and quick to detect the presence of any of a number of pathogens that are present in a sample.

### SUMMARY

A multiple microbe detection apparatus and methods based on array technology have been invented. A multiplex PCR system was developed which successfully detects microbes, such as pathogenic microbes. The device and methods of using the device may be automated, and may be provided in a single unit. As disclosed herein, nucleic acid probes for many microbes can be put into a single microarray and a standardized process can be used to examine the presence of one or more microbes in a given sample. Using the apparatus and methods disclosed herein, microbe detection may become a daily routine process for clinical diagnosis, pathogen surveillance and guidance for treatment.

In one embodiment of the invention, an apparatus for detecting the presence of a microbe in a sample comprises a substrate that has a plurality of microbe identification sites. The microbe identification sites may be provided in one or more discrete regions on or in the substrate. Each microbe identification site has a unique address indicative of the position of the microbe identification site on the substrate. The apparatus also includes a plurality of nucleic acids provided as groups of nucleic acid probes. The groups of nucleic acid probes are at unique microbe identification sites, and each group of nucleic acid probes is complementary to a target nucleic acid of a microbe in a sample. Hybridization of a target nucleic acid in the sample to a nucleic acid probe in the microbe detection region provides a detectable signal at one or more microbe identification sites. The nucleic acid probes of the apparatus preferably are complementary to genetic sequences of viral pathogens, such as respiratory viral pathogens. Examples of some respiratory viral or non-viral pathogens include, and are not limited to, adenoviruses, influenza A virus, influenza B virus, influenza C virus, parainfluenza 1, parainfluenza 2, parainfluenza 3, parainfluenza 4, mumps virus, respiratory syncytial virus, enterovirus, rhinovirus, rubella virus, coronavirus, chlamidia pneumonia, and mycoplasma pneumonia.

In another embodiment, an apparatus for detecting the presence of a pathogen in a sample comprises a nucleic acid probe disposed on a substrate that hybridizes to a target nucleic acid of a pathogen on a substrate, and comprises a nucleotide sequence selected from a group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, and SEQ ID NO: 91. The apparatus may include a plurality of nucleic acid probes provided in groups on the substrate where the nucleic acid probes of each group comprise a nucleotide sequence from a group of nucleotide sequences having a SEQ ID NO: 1-91.

In certain embodiments, the nucleic acid probes of the foregoing apparatus are complementary to a nucleotide sequence of a target nucleic acid that has at least 80% homology among different types of microbes in a microbe family. In additional embodiments, the homology may be greater than 90%, for example, greater than 95%, and may be about 98%.

The nucleic acid probes typically contain between 65 and 80 nucleotides, for example, the probes may comprise at least 70 nucleotides, or may comprise between 70 and 75 nucleotides. The nucleic acid probes are typically provided as single stranded (sense or antisense) nucleic acid molecules. In certain embodiments, each of the nucleic acid probes in a group have an identical nucleotide sequence. The nucleic acid probes may be printed or synthesized on the substrate.

The target nucleic acids used with the foregoing apparatus may be amplified nucleic acids obtained by polymerase chain reaction, or they may be nucleic acids obtained directly from a sample. The target nucleic acids include a label that emits a detectable signal under appropriate conditions. Fluorescent tags are examples of suitable labels, and one preferred tag is Cy3, which may be incorporated with a specific nucleotide that is incorporated into the amplified nucleic acids during the polymerase chain reaction.

The foregoing apparatus may be provided in a kit. One kit includes nucleic acid primers structured to hybridize to different regions of a target nucleic acid of a microbe for a polymerase chain reaction. The nucleic acid primers may be used as a single pair for a single microbe, or may be used in two or more pairs for multiple microbes. The kits of the invention may also include microbe microarray slides, a scanner and an analyzer to receive signals from the apparatus and to analyze the signals so received. The scanner and analyzer preferably include a computer due to the large amounts of data generated by the apparatus disclosed herein.

In certain embodiments of the invention, a nucleic acid primer for a polymerase chain reaction of a target nucleic acid of a microbe suitable for use in the foregoing kits may comprise a nucleotide sequence selected from a group consisting of: SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, and SEQ ID NO: 113. In additional embodiments, pairs of primers comprise the nucleotide sequences of SEQ ID NO: 92-113. The primers may each have a nucleotide sequence that is structured to hybridize to different regions of a target nucleic acid of a microbe.

In accordance with another embodiment of the invention, a method for detecting the presence of a microbe in a sample comprises: (i) identifying a target nucleic acid of a microbe in a sample; (ii) labeling the target nucleic acid; (iii) providing a substrate that has different groups of nucleic acid probes at different locations on the substrate; and (iv) exposing the labeled target nucleic acid to the substrate such that the labeled target nucleic acid will hybridize to nucleic acid probes that have a nucleotide sequence complementary to the nucleotide sequence of the labeled target nucleic acid. The target nucleic acids may be identified in a biological sample, which includes biological fluids and tissues. For example, the target nucleic acids for a microbe may be identified in a biological sample suspected of containing that pathogen. Examples of biological fluids that are useful in practicing the methods of the invention include, and are not limited to, blood, serum, mucus, urine, sputum, saliva, cerebral spinal fluid, and perspiration. The nucleic acids identified in the sample are amplified in certain embodiments, using at least one pair of nucleic acid primers, such as the primers of SEQ ID NO: 92-113, and a polymerase chain reaction. In additional embodiments, at least two pairs of nucleic acid primers are used. The labeling of the target nucleic acid preferably occurs before the target nucleic acid is exposed to the nucleic acid probes on the substrate, and may occur when the target nucleic acid is being amplified using PCR. The methods of the invention may also include a step of detecting a label at specific locations on the substrate where the labeled target nucleic acid hybridized to the nucleic acid probes. Detection steps of the methods may include steps of detecting a fluorescent signal.

### BRIEF DESCRIPTION OF FIGURES

FIG. 1 depicts an apparatus for detecting one or more microbes in a sample, as described herein.

FIG. 2 depicts a method for making an apparatus for detecting one or more microbes in a sample.

FIG. 3 depicts a method for detecting one or more microbes in a sample.

### DETAILED DESCRIPTION OF PRESENTLY PREFERRED EMBODIMENTS

An apparatus for detecting one or more microbes in a sample includes a substrate having a plurality of microbe identification sites. The microbe identification sites may be provided in one or more distinct detection regions of the substrate. The microbe identification sites are areas of the substrate that contains clusters or groups of nucleic acids or nucleic acid molecules having nucleic acid sequences that are complementary to nucleotide sequences of nucleic acids of a microbe. The nucleic acids that are obtained from a sample, such as a biological sample, which includes both solid and liquid samples obtained from a subject, including humans, are labeled and are exposed to the microbe identification sites of the substrate. The labeled target nucleic acids which have nucleotide sequences that are complementary to the nucleotide sequences of the nucleic acids of the microbe identification sites will hybridize to the nucleic acid probes of the microbe identificatin sites. The non-hybridized nucleic acids from the sample may be washed from the substrate, and a signal produced by the hybridized nucleic acids will be detected at discrete regions on the substrate. The presence and location of detectable signals indicate the presence of one or more particular microbes in the sample.

The apparatus and methods disclosed herein can be used for clinical diagnosis, research, epidemiological surveillance, bioterrorism countermeasures, environmental pathogen surveys, and monitoring of food contaminants, among other things. Depending on the nucleotide sequences of the nucleic acid probes of the apparatus, the apparatus can be used to detect pathogenic and non-pathogenic microbes. Although the specific examples herein are directed to detecting viral pathogens in a sample, the invention may be practiced and used to detect any pathogenic or non-pathogenic microbe in a sample. In certain embodiments, the microbe may be a viral pathogen, such as a virus or viroid. In other embodiments, the microbe may be a bacterial pathogen. In additional embodiments, the microbe may be a parasite, a fungus, or a yeast. In still further embodiments, the microbe may be pathogenic or non-pathogenic portions of microbes that contain one or more nucleic acids. The microbes may comprise cellular or acellular components that have nucleic acids. The apparatus and methods disclosed herein thus permit the detection of a vast number, e.g., several thousand, of microbes, such as pathogens, that may be present in a biological or environmental sample in a single device that is easy to use and provides rapid and reliable results. In addition, the apparatus and methods permit the detection of multiple types of microbes in one assay, e.g., the apparatus and methods may be used to detect bacterial and viral pathogens in one assay, non-pathogenic bacteria and viruses in one assay, or any other combination of various microbes, as identified above.

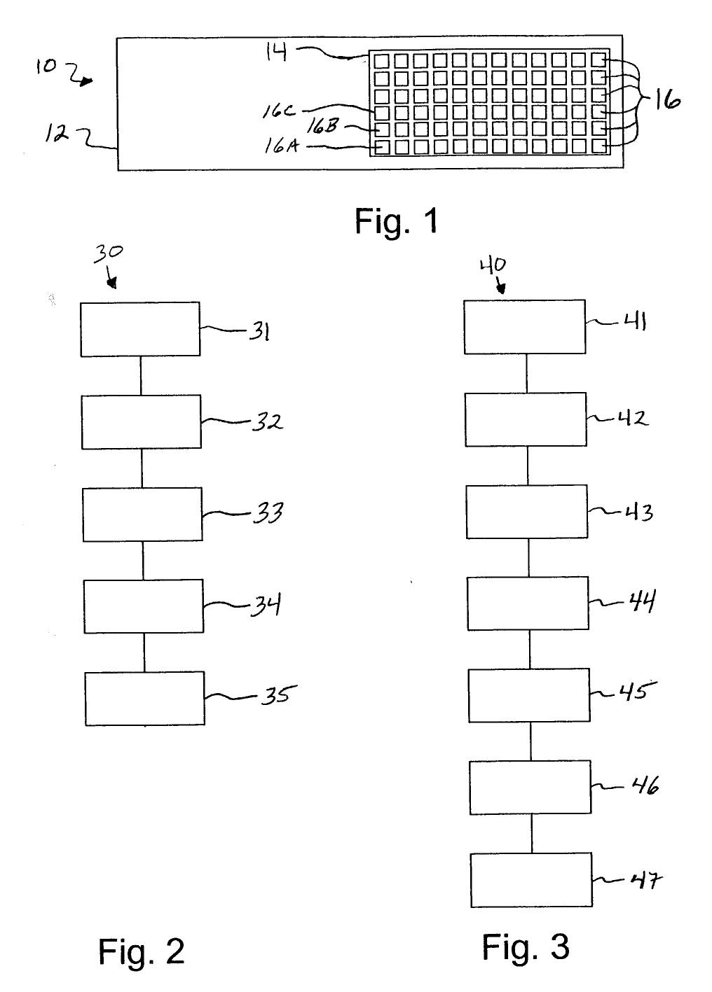

Referring to one embodiment of the invention, and more particularly to the embodiment illustrated in FIG. 1, an apparatus 10 for detecting a pathogen is illustrated as comprising a substrate 12, a pathogen detection region 14 located on the substrate, and a plurality of pathogen identification sites 16 located on the substrate. As depicted in FIG. 1, pathogen identification sites 16 are located in pathogen detection region 14. In accordance with this embodiment, apparatus 10 is a microarray of nucleic acid probes.

Substrate 12 may be made from any suitable material that permits, or can be modified to permit, nucleotides to be attached thereto. The substrate should be stable under various reaction conditions associated with nucleotide chemistry and in particular, nucleic acid hybridization. Suitable materials for the substrate include organic and inorganic materials, and are not limited to, glass materials as well as plastics, including polystyrene, polymethylmethacrylate, polycarbonate, polycyanoacrylate, polyurethane, and polyimides. In one embodiment, substrate 12 comprises a coated glass slide, such as a poly-lysine coated glass slide.

Although pathogen detection region 14 is illustrated as occupying only a portion of substrate 12, pathogen detection region 14 may be provided occupying more or less of the surface of substrate 12, for example a major portion or a minor portion of the surface of substrate 12. In certain embodiments, the apparatus is provided with a plurality of pathogen identification sites without a discrete pathogen detection region. In addition, pathogen detection region is illustrated only on one surface of substrate 12; however, other apparatus may include one or more pathogen detection regions on one or more surfaces of substrate 12. The number of pathogen identification sites 16 provided on the substrate 12 or in pathogen detection region 14 can vary from one to several thousand. As understood by persons of ordinary skill in the art, substrate 12 or pathogen detection region 14 may include a number of pathogen identification sites 16 that permit a sample to be assayed for multiple pathogens. The pathogen identification sites 16 can be relatively densely arranged on substrate 12 or in pathogen detection region 14 so long as the signal generated at any particular pathogen identification site is distinguishable from a signal generated at a nearby pathogen identification site, including adjacent pathogen identification sites. The pathogen identification sites typically range in number from 10 to 1,000,000 per pathogen detection region. In one embodiment, pathogen detection region 14 comprises at least 1 pathogen identification site per cm², but in more preferred embodiments, pathogen detection region comprises between 100 pathogen identification sites per cm² and between 100,000 pathogen identification sites per cm². As disclosed herein, each pathogen identification site 16 comprises a group of nucleic acid molecules, e.g., nucleic acid probes, that have a nucleotide sequence that is complementary to a nucleotide sequence, e.g., a genetic sequence, of a pathogen, and preferably a single pathogen. Thus, the pathogen identification sites 16 can be dimensioned and positioned in pathogen detection region 14 to achieve nucleic acid probe densities greater than 400 nucleotides per cm², e.g., as disclosed in U.S. Pat. No. 5,744,305. In more preferred embodiments, the substrate comprises a nucleic acid probe density of at least 1000 nucleotides per cm², e.g., as disclosed in U.S. Pat. No. 5,445,934. Examples of suitable microarrays used in accordance with the invention herein disclose include those disclosed in U.S. Pat. Nos. 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,624,711; 5,639,603; 5,658,734; 5,837,832; 5,663,242; 6,027,880; and 6,258,536; PCT Publication Nos. WO 93/17126; WO 95/11995; WO 95/35505; and European Pat. Nos. EP 742 287; and EP 799 897.

Each pathogen identification site 16 provided on substrate 12 can be distinguished from the other pathogen identification sites on substrate 12 based on their respective locations on the substrate. For example, each pathogen identification site 16 may have a specific address that indicates the position of the nucleic acid probes of that pathogen identification site on the substrate, or that indicates the position of the nucleic acid probes of that pathogen identification site to the other pathogen identification sites. As disclosed herein, the address of each pathogen identification site 16 is recorded in a computer so that when the pathogen detection region is scanned and analyzed for detectable signals, the position of the signal will be correlated to a nucleotide sequence of the probes contained at the pathogen identification site, and thus, the identity of the pathogen can be determined based on the position of the signal. For example, a Cartesian coordinate system may be used to provide a unique address to each pathogen identification site. Referring to FIG. 1, pathogen identification site 16A may have an address of (0,0), identification site 16B may have an address of (1,0); and identification site 16C may have an address of (2,0). The other pathogen identification sites could have unique addresses based on similar principles.

As indicated above, each pathogen identification site comprises a plurality of nucleic acids or nucleic acid probes. The probes are attached to substrate 12 so that the probes are fixed in position on the substrate. The probes may be attached to substrate 12 using any suitable process. One example includes synthesizing the probes on the substrate. In certain embodiments, Very Large Scale Immobilized Polymer Synthesis (VSLIPS™) methods may be used to attach the probes to the substrate, as is understood by persons of ordinary skill in the art. In particular, the probes may be attached using the methods disclosed in U.S. Pat. No. 5,134,854; 5,384,261; and/or 5,445,934. In other embodiments, the probes may be attached to the substrate by depositing presynthesized probes onto the substrate, for example, using the methods disclosed in PCT Publication No. WO 95/35505. The nucleic acid probes for the different pathogen genetic sequences may be provided on the substrate in duplex or triplex. Each duplex or triplex may represent a specific pathogen. Thus, hundreds or thousands of different probes for different pathogens can be provided on a single substrate, as indicated above.

The nucleic acid probes of each pathogen identification site have a nucleotide sequence that is complementary to and hybridizes to a nucleic acid of a pathogen, or a nucleic acid that has a nucleotide sequence, e.g., a genetic sequence, that is conserved within a family of types of pathogen. In one embodiment of the invention, the nucleic acid probes of a single identification site are complementary to a single pathogen. The nucleic acid probes of a single pathogen identification site preferably comprise a nucleotide sequence that is identical among the probes of the single pathogen identification site. However, nucleic acid probes may be provided in a single pathogen identification site that have sufficient homology to each other so that the probes at that single site hybridize to nucleic acids of one or more related pathogens. In certain embodiments, the nucleic acid probes will have at least eighty percent homology to each other in a single identification site. In preferred embodiments, the nucleic acid probes have at least ninety percent homology to each other, for example ninety-five percent homology. As indicated above, in one embodiment, the nucleic acid probes at a single identification site have the same nucleotide sequence.

The nucleic acid probes described herein are oliogonucleotides comprising between fifty and one hundred nucleotides. In certain embodiments, the nucleic acid probes are nucleic acids comprising between about 70 and about 75 nucleotides, for example between 66 nucleotides and 80 nucleotides. The nucleic acid probes are preferably single stranded nucleic acids and may comprise a sense strand, an anti-sense strand, or both.

The nucleic acid probes for a particular pathogen are selected based on the homology of nucleotide sequences for different strains or groups of pathogens. Nucleic acid probes may be synthesized to have a nucleotide sequence that is complementary to the conserved nucleotide sequence of the different pathogen strains.

As illustrated in FIG. 2, a method 30 for making an apparatus for detecting a pathogen in a sample comprises steps of: identifying (31) a nucleotide sequence or sequences for one or more pathogens; identifying (32) conserved nucleotide sequences among the different strains of a pathogen; designing (33) nucleotide primers that flank the conserved nucleotide sequences; synthesizing (34) nucleic acid probes based on the conserved nucleotide sequences; and applying (35) the nucleic acid probes to a substrate.

The nucleotide sequences obtained from one or more pathogens may be identified and obtained from a database containing nucleotide sequences, such as the nucleotide database from the National Center of Biotechnology Information (NCBI). Alternatively, the nucleotide sequences may be identified and obtained by extracting and sequencing nucleic acid molecules obtained from microbes, such as pathogens in a sample. Conserved nucleotide sequences are identified by aligning the nucleotide sequences of the pathogens using computer software, such as GCG software, as is conventionally practiced by those of ordinary skill in the art. The region or regions of the nucleotide sequences that are the most conserved, e.g., have the greatest homology, are selected as the conserved nucleotide sequences for primer design, as discussed herein. Accordingly, conserved nucleotide sequences having approximately ten percent homology may be used in primer design if that sequence corresponds to the region of the nucleic acid that has the highest homology among the various nucleic acids. However, it is more preferred that homologies of at least eighty percent are identified, and more preferably, sequence homologies of at least ninety percent, such as ninety-five percent are used to identify nucleotide sequences of interest. As indicated above, the conserved nucleotide sequences are used to design the primers, which may then be used in a polymerase chain reaction (PCR) to amplify the nucleotide sequences flanked by the primers, as is conventionally practiced. As understood by persons skilled in the art, it may be desirable to use degenerate primers for sequences that do not necessarily have optimal homologies. Examples of degenerate primers are provided in Table II, hereinbelow, with the translation of the degererate nucleotides provided in Table III. The amplified PCR products, amplicon, are then used as the nucleic acid probes of the apparatus. The amplicon may be directly deposited or printed on the substrate, as indicated above, or the nucleic acid probes may be chemically synthesized to have sequences that are identical, or nearly identical, to the amplicon, and then may be deposited on the substrate. Although the method illustrated in FIG. 2 utilizes PCR to generate amplicon as the nucleic acid probes, additional methods may omit this step, and synthetically generate nucleic acid probes based on the conserved nucleotide sequences.

Examples of various nucleic acid probes for viral respiratory pathogens generated using the methods disclosed herein are provided in Table I.

TABLE I

| | | | | | |
|---|---|---|---|---|---|
| | | | | Se- | |
| SEQ | | | | quence | |
| ID | | | Genbank | loca- | |
| NO: | Identity | Strain | # | tion | Sequence, 5--3′ |

| | | | | | |
|---|---|---|---|---|---|
| 1 | Polio- | mahoney | V01149 | 465- | tccgccacggacttg |
| | virus 1 | | | 535 | cgcgttacgacaggc |
| | | | | | caatcactggtttgt |
| | | | | | gaccacctgctccga |
| | | | | | ggttgggatt |
| 2 | PV3 | turtey | L76410 | 468- | tccgccacagacttt |
| | | | | 538 | cgcgttacgacaggc |
| | | | | | aaaccactggtttgt |
| | | | | | gaccacctgctccgt |
| | | | | | ggttgggatt |
| 3 | PV2 | usa | L76412 | 468- | gttccgccacggact |
| | | | | 538 | tgcgcgttacgacaa |
| | | | | | gccaatcactggttc |
| | | | | | gtgaccgcttgctcc |
| | | | | | gtggttagga |
| 4 | Entero | na | Nc001430 | 468- | tccgccacagacttg |
| | 70 | | | 538 | cgcattacgacaaac |
| | | | | | cactcactggattgt |
| | | | | | gagcatttgctccgt |
| | | | | | ggttgggatt |
| 5 | Cox- | | Nc001429 | 472- | tccgccacggacttg |
| | sackie | | | 542 | cgcgttacgacaggc |
| | A24 | | | | tggctgctggattgc |
| | | | | | aactacctgctccat |
| | | | | | ggttaggatt |
| 6 | Entero | Tw1929/ | Af117630 | 372- | ccgctgcagagttgc |
| | 71 | 89 | | 442 | ccgttacgacacacc |
| | | | | | actcactggtttgtg |
| | | | | | agcatgtgctccgca |
| | | | | | gttgggatta |
| 7 | Rhino 87 | | Af108187 | 471- | tccgccacggacttg |
| | | | | 541 | cgcgttacgacaagc |
| | | | | | aacccactggtttgt |
| | | | | | gagcacttgctccat |
| | | | | | ggttaggatt |
| 8 | Echo30 | | Aj | 471- | tccgctgcagagttg |
| | | | 131523 | 541 | cccgttacgacaggc |
| | | | | | tactcactggtctgt |
| | | | | | gagcacctgctccgc |
| | | | | | agttaggatt |
| 9 | Echo 6 | Lytic, | Nc001657 | 468- | tccgctgcagagttg |
| | | Charlies | | 538 | cccgttacgacaggc |
| | | | | | cacccactggattgt |
| | | | | | gagcacctgctccgc |
| | | | | | agttaagatt |
| 10 | Echo 4 | | X89534 | 353- | tccgctgcggagtta |
| | | | | 423 | cccattacgacacac |
| | | | | | cactcactggcttgt |
| | | | | | gagcgtgtgctccgc |
| | | | | | agttaggatt |
| 11 | Echo 3 | Morrisey | X89533 | 353- | tccgctgcagagtta |
| | | | | 423 | cccgttacgacagcc |
| | | | | | tgcccactggattgt |
| | | | | | gagtacttgctccgc |
| | | | | | agttaggatt |
| 12 | Ev | 96-83csf | Nc002472 | 475- | tccgctgcagagttg |
| | Yanbian | | | 545 | cccgttacgacatgc |
| | | | | | caccctctggattga |
| | | | | | gggcacatgctccgc |
| | | | | | agttaggatt |
| 13 | Coxs A21 | Coe | Nc001428 | 463- | tccgccacagactta |
| | | | | 533 | cgcattacgacaacc |
| | | | | | tactcactgggtcgt |
| | | | | | gagcgattgctccgt |
| | | | | | ggttaggatt |
| 14 | Echo 25 | M1262 | X90723 | 469- | tccgccgcagagtta |
| | | | | 539 | cccattacgacaggt |
| | | | | | tgcccactggtttgt |
| | | | | | gggtgcctgctccga |
| | | | | | gattaggatt |
| 15 | Echo 8 | bryson | X89539 | 351- | tccgctgcagagttg |
| | | | | 421 | cccgttacgacacgc |
| | | | | | caccctctgggttga |
| | | | | | gagcacgtgctccgc |
| | | | | | agttaggatt |
| 16 | Echo2 | | X89532 | 353- | ccgctgcagagttgc |
| | | | | 423 | ccattacgacaggct |
| | | | | | gcccactggctcgtg |
| | | | | | ggtacctgctccgca |
| | | | | | gttaggatta |
| 17 | Cox B6 | china | Af225476 | 428- | tccgccgcagagttg |
| | | | | 498 | cccgttacgacagac |
| | | | | | tgcccactggtgtgt |
| | | | | | gggtgtctgctccgc |
| | | | | | ggttaggatt |
| 18 | CoxB2 | China | Af225474 | 427- | tccgctgcagagttg |
| | | | | 497 | cccgttacgacacgc |
| | | | | | catcctctggattga |
| | | | | | ggtcgcgtgctccgc |
| | | | | | agttgggatt |
| 19 | Echo 11 | | X80059 | 471- | ccgctgcagagttgc |
| | | | | 541 | ccgttacgacacact |
| | | | | | gccccttggattagg |
| | | | | | ggtatgtcgtccgca |
| | | | | | gttaggatta |
| 20 | Cox B5 | Faulkner | Af114383 | 470- | tccgctgcagagttg |
| | | | | 540 | cccgttacgacacgc |
| | | | | | caccccctggaatgg |
| | | | | | aggcacgtgctccgc |
| | | | | | agttaggatt |
| 21 | Echo 9 | Amc3 | U77070 | 466- | tccgctgcagagtta |
| | | | | 536 | cccgttacgacagac |
| | | | | | tgcccactggcttgt |
| | | | | | gggtgtctgctccgc |
| | | | | | agttgggatta |
| 22 | Cox A9 | Griggs | Nc002347 | 470- | tccgctgcagagttg |
| | | | | 540 | cccgttacgacacgc |
| | | | | | tgccccctggtttga |
| | | | | | gggtgcgtgctccgc |
| | | | | | agttgggatt |
| 23 | Cox B4 | JVBB | Nc001360 | 470- | tccgctgcagagttg |
| | | | | 540 | cccgttacgacacac |
| | | | | | cactcgctggcttgc |
| | | | | | gaacgtgtgctccgc |
| | | | | | agttaggatt |
| 24 | Ev | | Af132497 | 223- | tccgctgcagagttg |
| | stutt- | | | 293 | cccgttacgacagac |
| | gart | | | | tactcgctggtttgc |
| | | | | | tagcgtctgctccgc |
| | | | | | agttaggatt |
| 25 | Echo 12 | | Nc001810 | 468- | tccgctgcagagttg |
| | | | | 538 | cccattacgacaagc |
| | | | | | cacccactgggttgt |
| | | | | | gggcacttgctccac |
| | | | | | agttaggatt |
| 26 | Cox A16 | | Nc001612 | 474- | tccgctgcagagttg |
| | | | | 544 | cccgttacgacacac |
| | | | | | tgccccctgggtcga |
| | | | | | gggtatgtgctccgc |
| | | | | | agttaggatt |
| 27 | Cox B3 | | U30927 | 165- | tccgctgcagagtta |
| | | | | 235 | cccgttacgacacac |
| | | | | | tacccactggtttgt |
| | | | | | gggcatgtgctccgc |
| | | | | | agttaggatt |
| 28 | Echo5 | | X89535 | 353- | ccgctgcagagttac |
| | | | | 423 | ccattacgacaggct |
| | | | | | gcccactggctcgtg |
| | | | | | ggtgcctgctccgca |
| | | | | | gttaggatt |
| 29 | Porcine | | Nc001827 | 463- | ccgccacagagttgc |
| | ev 9 | | | 533 | ccgttacgacgccct |
| | | | | | gccagctggattgct |
| | | | | | ggtggacgctccgtg |
| | | | | | gttaggatta |
| 30 | Cox b6 | schmitt | Af114384 | 469- | tccgctgcggagttg |
| | | | | 539 | cccattacgacaagc |
| | | | | | tgctcgctggtctgc |
| | | | | | gagtgcctgctccgc |
| | | | | | agttaggatt |
| 31 | Echo 7 | | L76400 | 434- | tccgctgcagagttg |
| | | | | 504 | cccgttacgacagac |
| | | | | | tgcccactggctgtg |
| | | | | | ggtatctgctccgca |
| | | | | | gttaggatt |
| 32 | Echo 1 | | Af029859 | 470- | tccgctgcagagtta |
| | | | | 540 | cccattacgacacac |
| | | | | | tgctccctggattgg |
| | | | | | gagtatgtgctccgc |
| | | | | | agttaggatt |
| 33 | Echo 27 | bacon | L76396 | 446- | ccgctgcagagttgc |
| | | | | 516 | ccgttacgacacaca |
| | | | | | ccacggttgtgggca |
| | | | | | tgtgctccgcagtta |
| | | | | | ggatta |
| 34 | HRV | 95-04967 | Af108177 | 412- | tccgtcccacagttg |
| | (Human | | | 482 | cccattacgaccaac |
| | rhino- | | | | tacgcattggtttat |
| | virus) | | | | gcgcattggatgtgg |
| | | | | | ggttggatt |
| 35 | Ev sp | ENT/ | Aj295041 | 401- | tccgctgcagagttg |
| | | 00/15 | | 471 | cccattacgacagaa |
| | | 394 | | | tacccgctggcgtgc |
| | | | | | gggcatctgctccgc |
| | | | | | agttgggatt |
| 36 | HRV | HRV14 | K02121 | 475- | cccgtcccggaattg |
| | | | | 545 | ctcattacgacctta |
| | | | | | caaccactggatcgt |
| | | | | | ggcataaggctctag |
| | | | | | ggttaaggtt |
| 37 | hrv | Hrv 1B | Nc001435 | 466- | cccatcccgcaattg |
| | | | | 536 | ctcattacgaccata |
| | | | | | agctcattggtttat |
| | | | | | gagccatggctgcag |
| | | | | | gtttaaggtt |
| 38 | Bovine | K2577 | Af123432 | 542- | tccgcctccaactta |
| | EV | | | 612 | cgcattacgacgtag |
| | | | | | caacactgggttgtg |
| | | | | | cgcacacgctcggag |
| | | | | | gttgggatt |
| 39 | Cox B1 | | M16560 | 468- | tccgctgcagagttg |
| | | | | 538 | cccgttacgacacac |
| | | | | | tgccccctggtttga |
| | | | | | gggtatgtgctccgc |
| | | | | | agttaggatt |
| 40 | Polio I | Mahoney | V01149 | 7180- | aagctaaaaggcaca |
| | | | | 7250 | gagagcgaacgtgat |
| | | | | | cctgagtgttcctag |
| | | | | | gatctttagtccatc |
| | | | | | taattgattc |
| 41 | Polio 3 | PV Sabin | X00596 | 7173- | agccaatagacacaa |
| | | 3 | | 7243 | ggagcgtacatggtc |
| | | | | | ctgcgtattccgagg |
| | | | | | atcttttgtccatct |
| | | | | | gattgattca |
| 42 | Ev 70 | | Nc001430 | 7140- | aagtttcttcaccat |
| | | | | 7210 | tgtgccaggcagtag |
| | | | | | gcacaaggatctcac |
| | | | | | atgatcttgtgtgtt |
| | | | | | tctcggatct |
| 43 | Pv 1 | mahoney | V01149 | 7371- | ctccgaattaaagaa |
| | | | | 7440 | aaatttacccctaca |
| | | | | | acagtatgacccaat |
| | | | | | ccaattcgactgagg |
| | | | | | tagggttact |
| 44 | rhino- | Hrv 14 | K02121 | 7143- | ataaactcctacttc |
| | virus | | | 7213 | tactcaaattaagtg |
| | | | | | tctatattgttaacc |
| | | | | | taaaagaggtccaac |
| | | | | | cagcgcctcc |
| 45 | Human | H. | NM- | 901- | cagcactgtgttggc |
| | beta- | sapien | 001101 | 969 | gtacaggtctttgcg |
| | actin | | | | gatgtccacgtcaca |
| | | | | | cttcatgatggagtt |
| | | | | | gaaggtagtt |
| 46 | Human | H. | Af261085 | 601- | atccacagtcttctg |
| | GAPDH | sapien | | 669 | ggtggcagtgatggc |
| | | | | | atggactgtggtcat |
| | | | | | gagtccttccacgat |
| | | | | | accaaagttg |
| 47 | h. | H. sapien | X52856 | 301- | gatggacttgccact |
| | cycphil- | | | 369 | agtgccattatggcg |
| | in | | | | tgtgaagtcaccacc |
| | | | | | ctgacacataaaccc |
| | | | | | tggaataatt |
| 48 | Human | H. | X54156 | 12061- | gcctctggcattctg |
| | P53 | sapien | | 12129 | ggagcttcatctgga |
| | | | | | cctgggtcttcagtg |
| | | | | | aaccattgttcaata |
| | | | | | tcgtccgggg |
| 49 | Human | H. | Nm- | 61-129 | gcaggccttcagtca |
| | Sod1 | sapien | 000454 | | gtcctttaatgcttc |
| | | | | | cccacaccttcactg |
| | | | | | gtccattactttcct |
| | | | | | tctgctcgaa |
| 50 | Human | H. | Nm- | 901- | gaggaccacgtgggt |
| | Nos1 | sapien | 000620 | 969 | ctcagaggcaatgcc |
| | | | | | tctgagtacctccag |
| | | | | | ggcgctgtcatagct |
| | | | | | caggtccacc |
| 51 | adeno- | Type2 | Nc_{—} | 8803- | ggccgaaatcgccta |
| | virus | | 001405 | 8877 | tcaagacaactcagg |
| | | | | | agacgtgcaggagat |
| | | | | | tttgcgccaggccgc |
| | | | | | cgtcaacgacaccg |
| 52 | adeno- | Type2 | Nc_{—} | 8878- | tccaacgacctcgcc |
| | virus | | 001405 | 8951 | gccaccgtggagcga |
| | | | | | gccggacgcggagat |
| | | | | | ctccaggaggaagag |
| | | | | | atcgagcagttcat |
| 53 | adeno- | Type4 | X74508 | 1624- | ccgagattgcctacc |
| | virus | | | 1697 | aggacaactcgggcg |
| | | | | | acgtgcaagagattc |
| | | | | | tgcggcaggccgccg |
| | | | | | tcaatgataccgag |
| 54 | adeno- | Type 17 | Nc_{—} | 8538- | cgagatcgcctacca |
| | virus | | 002067 | 8612 | ggacaactcgggcga |
| | | | | | cgtgcaagagatcct |
| | | | | | gcgtcagcccgccgt |
| | | | | | caatgacgccgaga |
| 55 | adeno- | Type 7 | X03000 | 8651- | cgagatcgcctacca |
| | virus | | | 8723 | ggacaactctggcga |
| | | | | | cgtgcaagaaatcct |
| | | | | | taggcaagccgccgt |
| | | | | | caacgataccgag |
| 56 | adeno- | Type40 | Nc_{—} | 8333- | ggagattgcctacca |
| | virus | | 001454 | 8406 | agacaactctggcga |
| | | | | | cgtgcaggagattct |
| | | | | | gcgacaagccgcggt |
| | | | | | caacgatgccgata |
| 57 | adeno- | Type 12 | Nc_{—} | 8539- | agaaattgcctacca |
| | virus | | 001460 | 8612 | agataactcaggcga |
| | | | | | cgttcaggagatttt |
| | | | | | aagacaggctgcagt |
| | | | | | aaatgacactgaga |
| 58 | measles | Edmoston | Nc_{—} | 6481- | gtgtgcagccaaaat |
| | | | 001498 | 6554 | gccttgtacccgatg |
| | | | | | agtcctctgctccaa |
| | | | | | gaatgcctccggggg |
| | | | | | tccaccaagtcctg |
| 59 | measles | Edmoston | Nc_{—} | 6555- | tgctcgtacactcgt |
| | | | 001498 | 6628 | atccgggtcttttgg |
| | | | | | gaaccggttcatttt |
| | | | | | atcacaagggaacct |
| | | | | | aatagccaattgtg |
| 60 | PIV 1 | Washing- | AF016280 | 631- | tatatgcgtattcat |
| | | ton, | | 704 | caaacttaatcactc |
| | | 1964 | | | aaggatgtgcagata |
| | | | | | tagggaagtcatatc |
| | | | | | aggttttacaatta |
| 61 | PIV3 | | Nc_{—} | 8051- | ggtaacaagatctat |
| | | | 001796 | 8024 | atatatacaagatct |
| | | | | | acaagttggcatagc |
| | | | | | aarttacaattagga |
| | | | | | ataattgatattac |
| 62 | PIV 4B | 68-333 | D49822 | 1467- | cgaccgatttaaatc |
| | | | | 1540 | aatataatcaattac |
| | | | | | tcaagagtgctgaga |
| | | | | | accacatccaacggt |
| | | | | | caaatgattactta |
| 63 | PIV 4A | M-25 | D49821 | 1467- | caactgatttaaatc |
| | | | | 1540 | aatacaatcaattac |
| | | | | | tcaagagtgctgaag |
| | | | | | atcacatccaacgat |
| | | | | | caactgattactta |
| 64 | PIV2, | | X57559 | 12461- | tagaacagaggaaag |
| | | | | 12534 | aagagttgcatcaat |
| | | | | | ggcatatattaaagg |
| | | | | | tgccacacacagttt |
| | | | | | gaaggctgctctta |
| 65 | mumps | Jeryl | Af201473 | 12111- | ccaaaacagatgaac |
| | | Lynn | | 12184 | gaagggttgcatcaa |
| | | | | | tggcttatatcaaag |
| | | | | | gggcatcagtatcac |
| | | | | | ttaaatcagcactc |
| 66 | mumps | Miya- | Nc_00220 | 12111- | ctaaaacagatgarc |
| | | hara/ | 0/ | 12184 | ggagggttgcgtcaa |
| | | Glouc1/ | af28079 | | tggcttacatcaaag |
| | | UK | | | gagcatctgtatcac |
| | | 96 | | | ttaaatcagcactc |
| 67 | RSV | | M74568 | 6881- | cacatctctaggagc |
| | | | | 6954 | cattgtgtcatgcta |
| | | | | | tggcaaaactaaatg |
| | | | | | tacagcatccaataa |
| | | | | | aaatcgtggaatca |
| 68 | RSV | B1 | NC_{—} | 6886- | tacttctcttggagc |
| | | | 001781 | 6959 | tatagtgtcatgcta |
| | | | | | tggtaaaactaaatg |
| | | | | | cactgcatccaacaa |
| | | | | | aaatcgtgggatta |
| 69 | Influ- | HK/498/ | Af255370 | 101- | cagagacaagaagat |
| | enza A | 97, H3N2 | | 174 | gtctttgcagggaaa |
| | | | | | aacactgatcttgag |
| | | | | | gctctcatggaatgg |
| | | | | | ctaaagacaaaacc |
| 70 | Influ- | Guang- | L18999 | 101- | cagagacttgaagat |
| | enza A | dong/39/ | | 174 | gtctttgctgggaaa |
| | | 89, H3 | | | aacacmgatcttgag |
| | | 92 | | | gctctcatggaatgg |
| | | | | | ctaaagacaagacc |
| 71 | Influ- | Lenin- | M81582 | 101- | cagagacttgaagat |
| | enza A | grad, | | 174 | gtctttgctgggaag |
| | | 134/47/5 | | | aacacmgatcttgag |
| | | 7, H2N2 | | | gctctcatggagtgg |
| | | | | | ctaaagacaagacc |
| 72 | Influ- | Gull/ | M63538 | 101- | cagagacttgaagat |
| | enza A | Ma26/80, | | 174 | gtctttgcagggaaa |
| | | H13N6 | | | aacacmgaccttgaa |
| | | | | | gcactcatggaatgg |
| | | | | | ctaaagacaagacc |
| 73 | Influ- | Budgri- | M63536 | 101- | cagagacttgaagat |
| | enza A | gar/H./ | | 174 | gtttttgctggaaag |
| | | 1/77. | | | aatactgacctcgag |
| | | H4N6 | | | gctctcatggaatgg |
| | | | | | ctaaagacaagacc |
| 74 | Influ- | Shiga/ | AB036879 | 251- | caggaatgggaacaa |
| | enza B | 51/98 | | 324 | cagcaacaaaaaaga |
| | | | | | aaggcctgattctag |
| | | | | | ctgagagaaaaatga |
| | | | | | gaagatgtgtgagc |
| 75 | Influ- | Lee/40 | J02094 | 275- | caggaatgggaacaa |
| | enza B | | | 348 | cagcaacaaagaaga |
| | | | | | aaggcctaattctag |
| | | | | | ctgagagaaaaatga |
| | | | | | gaagatgtgtaagc |
| 76 | Influ- | Nara/2/ | AB000727 | 106- | agcctgcaaatcagc |
| | enza C | 85 | | 179 | agctaaactgatyaa |
| | | | | | gaatgaacatcttcc |
| | | | | | cytaatgtctggaga |
| | | | | | agccaccacaatgc |
| 77 | rhino- | Type 16 | NC_{—} | 461- | aaccttaaacctgca |
| | virus | | 001752 | 534 | gccagtgcacacaat |
| | | | | | ccagtgtgtagctgg |
| | | | | | tcgtaatgagcaatt |
| | | | | | gcgggatgggacca |
| 78 | rhino- | 95-03504 | AF108175 | 400- | aaccttaaccctgca |
| | virus | | | 473 | gctagagcgcgcaaa |
| | | | | | ccagcgtgtttctag |
| | | | | | tcgtaatgagcaatt |
| | | | | | gcgggatgggacc |
| 79 | rhino- | Type 29 | AF108181 | 399- | aaccttaaccctgca |
| | virus | | | 471 | gctagtgcatgcaat |
| | | | | | ccagcatgttgctag |
| | | | | | tcgtaatgagcaatt |
| | | | | | gcgggatgggacc |
| 80 | rhino- | Type 21 | AF108180 | 400- | aaccttaaccctgca |
| | virus | | | 472 | gctagtgcatgtaat |
| | | | | | ccaacatgttgctag |
| | | | | | tcgtaatgagcaatt |
| | | | | | gcgggacgggacc |
| 81 | rhino- | Type 1B | D00239 | 467- | aaccttaaacctgca |
| | virus | | | 540 | gccatggctcataaa |
| | | | | | ccaatgagcttatgg |
| | | | | | tcgtaatgagcaatt |
| | | | | | gcgggatgggaccg |
| 82 | rhino- | Type 58 | AF108183 | 404- | aaccttaaccccgca |
| | virus | | | 478 | gccgctgcccatgga |
| | | | | | tccagtgggtatacg |
| | | | | | gtcgtaacgcgcaat |
| | | | | | gtggggatgggacca |
| 83 | rhino- | 95-01821 | AF108169 | 404- | aaccttaaacccgca |
| | virus | | | 477 | gccaaggtgtgcaag |
| | | | | | ccagcatattcttgg |
| | | | | | tcgtaacgagcaatt |
| | | | | | gtgggatgggacca |
| 84 | rhino- | Type 87 | AF108187 | 407- | aatcctaaccatgga |
| | virus | | | 480 | gcaagtgctcacaaa |
| | | | | | ccagtgggttgcttg |
| | | | | | tcgtaacgcgcaagt |
| | | | | | ccgtggcggaaccg |
| 85 | rhino- | 94-09389 | AF108160 | 412- | aaccttaaacccgca |
| | virus | | | 485 | gccatggttcataaa |
| | | | | | ccaatgagcttatgg |
| | | | | | tcgtaatgagcaatt |
| | | | | | gtgggatgggaccg |
| 86 | Corona- | 229E | NC_{—} | 13071- | caaaggagttgttgg |
| | virus | | 002645 | 13144 | tgttttgaccttaga |
| | | | | | caaccaagatcttaa |
| | | | | | tgggaatttctatga |
| | | | | | cttcggtgacttt |
| 87 | Rubella | | NC_{—} | 8651- | ccaccgacaccgtga |
| | | | 001545 | 8724 | tgagcgtgttcgccc |
| | | | | | ttgctagctacgtcc |
| | | | | | agcaccctcacaaga |
| | | | | | ccgtccgggtcaag |
| 88 | | 379 | AB003340 | 401- | ccaccgacaccgtga |
| | | | | 474 | tgagtgtgttcgccc |
| | | | | | tcgccagctacgtcc |
| | | | | | agcacccccacaaga |
| | | | | | ccgtcagggtcaag |
| 89 | Myco- | Atcc2934 | AF132741 | 281- | aatgggactgagaca |
| | plasms- | 2 | | 354 | cggcccatactccta |
| | pneu- | | | | cgggaggcagcagta |
| | moniae | | | | gggaatttttcacaa |
| | | | | | tgagcgaaagcttg |
| 90 | Chlamy- | TW183 | L06108 | 291- | cgtctaggcggattg |
| | dia | | | 364 | agagattgaccgcca |
| | pneu- | | | | acactgggactgaga |
| | moniae | | | | cactgcccagactcc |
| | | | | | tacgggaggctgca |
| 91 | parvo- | HV | AF162273 | 3831- | ttgggtatactttcc |
| | virus | | | 3904 | ccctcaatatgctta |
| | | | | | cttaacagtaggaga |
| | | | | | tgttaacacacaagg |
| | | | | | aatctctggagaca |

The nucleic acid probes of the invention (SEQ ID NO: 1-91) are directed to viruses that include adenoviruses, influenza A virus, influenza B virus, influenza C, parainfluenza 1, parainfluenza 2, parainfluenza 3, parainfluenza 4, mumps virus, respiratory syncytial virus, enteroviruses and rhinovirus, rubella virus, coronavirus and two non virus pathogens chlamidia pneumonia and mycoplasma pneumonia.

In one embodiment of the invention, an apparatus for detecting one or more pathogens in a sample comprises a substrate having a pathogen detection region that includes a plurality of nucleic acid probes, each probe having a nucleotide sequence selected from the group consisting of SEQ ID NO: 1-91. Certain apparatus may include nucleic acid probes that consist essentially of the nucleic acid probes having nucleotide sequences of SEQ ID NO: 1-91.

Additional apparatus of the invention include a substrate having a pathogen detection region which comprises all of the nucleic acid probes having nucleotide sequences of SEQ ID NO: 1-91. These and additional apparatus thus provide a device that can detect one or more pathogens, such as respiratory viral pathogens in a sample, in one assay. Additional nucleic acid probes for the apparatus disclosed herein may be developed using the methods disclosed herein without departing from the spirit of the invention.

A method 40 of detecting a pathogen in a sample is illustrated in FIG. 3. Method 40 comprises the steps of: designing (41) nucleic acid primers that flank nucleotide sequences that are conserved among different strains or groups of pathogens; extracting (42) nucleic acids from a sample believed to contain one or more pathogens; reverse transcribing (43) any RNA into complementary DNA (cDNA); amplifying (44) the nucleic acids extracted from the sample; labeling (45) the nucleic acids with a label that can produce a detectable signal; exposing (46) the labeled target nucleic acids to a substrate containing a plurality of nucleic acid probes, such as substrate 12, disclosed hereinabove, and scanning and analyzing (47) the substrate for one or more detectable signals. The nucleic acid primers may range in size from about 20 nucleotides to about 25 nucleotides.

Although method 40 comprises the steps indicated above, the method may also include one or more additional steps, or may be practiced by combining or separating one or more steps. For example, the method may also include a step of purifying the nucleic acidss extracted from the sample. This may be particularly necessary when the sample is a biological sample, such as a blood sample, or a sample from bodily tissue. The method may also incorporate the labeling step into the amplification step. For example, one or more labeled nucleotides may be used during the PCR reaction so that the amplified nucleic acids incorporate the labeled nucleotides. Any suitable label may be used in labeling the target nucleic acids; for example, the nucleotides may be labeled with fluorescent tags, chemiluminescent tags, chromogenic tags, and/or spectroscopic tags. Some specific examples of fluorescent tags include fluorescein isothiocyanate, rhodamine, a fluorescent protein, phycoerythrin, Cy3, and the like. Other labels include; enzymes whose products are detectable (e.g., luciferase, beta-galactosidase, and the like); a cyanine dye; fluorescence-emitting metals, e.g., ¹⁵²Eu, or others of the lanthanide series, chemiluminescent compounds, e.g., luminol, isoluminol, acridinium salts, and the like; bioluminescent compounds, e.g., luciferin, aequorin (green fluorescent protein), and the like. Other examples of fluorescent labels include, but are not limited to, fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyflu-orescein (JOE), 6-carboxy-X-rhodamine (ROX), 6-carboxy-2′,4′,7′,4,7-hexach-lorofluorescein (HEX), 5-carboxyfluorescein (5-FAM) or N,N,N′,N′-tetramethyl-6carboxyrhodamine (TAMRA). Radioactive labels include, but are not limited to, ³²P, ³⁵S, ³H, and the like. In addition, the scanning and analyzing steps may be performed by a single device, such as a computer, or may be performed by a separate scanner and analyzer.

Exposing the labeled target nucleic acids to the substrate causes the target nucleic acids to hybridize to the nucleic acid probes provided on the substrate. The target nucleic acid molecules may be applied to the pathogen detection region of the substrate using any suitable method and device so long as the target nucleic acids are dispersed over the entire pathogen detection region to permit hybridization to occur between complementary nucleic acid probes and the target nucleic acids. Hybridization is well understood by persons of ordinary skill in the art, and refers to the association of two nucleic acid sequences to one another by hydrogen bonding, usually on opposite nucleic acid strands. As understood by persons of ordinary skill in the art, the degree of hybridization between any two nucleic acid molecules can vary depending on a number of factors, including the type and volume of solvent, reaction temperature, time of hybridization, agitation, blocking agents, concentration of the nucleic acid molecules, additional compounds or agents that affect the rate of association of sequences (e.g., dextran sulfate or polyethylene glycol), and the stringency of the washing conditions after hybridization. Stringency refers to conditions in a hybridization reaction that favor association of similar sequences of sequences that differ. Conditions that increase stringency of a hybridization reaction are widely known and published in the art. See, for example, Sambrook et al., Molecular Cloning: A Laboratory Manual, 3^{rd} Edition, 2001. Examples of relevant conditions include (in order of increasing stringency): incubation temperatures of 25° C., 37° C., 50° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water. One non-limiting example of stringent conditions are hybridization and washing at 50° C. or higher and in 0.1×SSC (9 mM NaCl/0.9 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5× Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions. Other stringent hybridization conditions are known in the art and may also be employed to identify nucleic acids of this particular embodiment of the invention.

Examples of the nucleic acid primers used in accordance with the invention disclosed herein are provided in Table II.

TABLE II

| | |
|---|---|
| SEQ ID | |
| NO: | Sequence, 5′-3′ |

| | |
|---|---|
| 92 | cctgaaagavagttcvacaga |
| 93 | thtacagccgmgtstggaacga |
| 94 | accttcattatcaattggtga |
| 95 | tcctgttgtcgttgatgtcata |
| 96 | ccacccatcagagtdccwta |
| 97 | ccgaawgcccawatatatac |
| 98 | ccrtcaggccccctcaaagccga |
| 99 | aaadcgtctacghtgcagtcc |
| 100 | gaatggataaaaaacaaaagatgc |
| 101 | tggccttctgctatttcaaatgc |
| 102 | tcctccggcccctgaatgygg |
| 103 | cacggwcacccaaagtagtyggt |
| 104 | ggtaaaagcccaccaaggcga |
| 105 | gaaagtgctttacaaccctaag |
| 106 | yttaaccagcaaagtgttaga |
| 107 | aggtgtwgttacacctgcat |
| 108 | cagatgtatatcaactgtgttc |
| 109 | tccctggtccaacagatgggt |
| 110 | gtgatacaaaaacagcatatgt |
| 111 | tccaatgtccagctaaattag |
| 112 | tgcatgcttccttggcatcat |
| 113 | gctccttcacctatgaatgct |

Referring to the nucleic acid sequences disclosed in Tables I and II, the following letters in Table III refer to the following nucleotide bases:

TABLE III

| Letter | Nucleotide |
|---|---|
| B | c, g, t |
| D | a, g, t |
| H | a, c, t |
| K | g, t |
| M | c, a |
| N | g, a, t, c |
| R | g, a |
| S | c, g |
| V | a, c, g |
| W | a, t |
| Y | c, t |
| I | inosine |

The primers disclosed in Table II were developed and used in pairs. For example, SEQ ID NO: 92 and 93 were used as a pair from the adenovirus sequence (GenBank # NC_{—}001405) to create a 238 nucleotide amplicon. SEQ ID NO: 92 was generated to nucleotides 8774-8794 of the sense strand of NC_{—}001405, and SEQ ID NO: 93 was generated to nucleotides 8990-9012 of the anti-sense strand NC_{—}001405. SEQ ID NO: 94 and 95 are primer pairs from the parainfluenza 1 sequence (GenBank # AF016280) used to create a 181 nucleotide amplicon. SEQ ID NO: 94 was generated to nucleotides 605-625 of the sense strand of AF016280; and SEQ ID NO: 95 was generated to nucleotides 765-786 of the anti-sense strand of AF016280. SEQ ID NO: 96 and 97 are a pair of primers from the mump and parainfluenza 2 L gene (GenBank # X57559). SEQ ID NO: 96 was generated to nucleotides 12432-12452 of the sense strand of X57559, and SEQ ID NO: 97 was generated to nucleotides 12546-12565 of the antisense strand of X57559. SEQ ID NO: 98 and 99 are pairs for the influenza A virus M gene (GenBank # AF255370). SEQ ID NO: 98 was generated to nucleotides 71-94 of the sense strand of AF255370; and SEQ ID NO: 99 was generated to nucleotides 242-262 of the antisense strand of AF255370. SEQ ID NO: 100 and 101 are pairs for the influenza B matrix gene (GenBank # AB036879). SEQ ID NO: 100 was generated to nucleotides 132-155 of the sense strand of AB036879; and SEQ ID NO: 101 was generated to nucleotides 334-354 of the antisense strand of AB036879. SEQ ID NO: 102 and 103 are pairs for enterovirus and rhinovirus (GenBank Nos. X80059 and NC_{—}001752). SEQ ID NO: 102 was generated to nucleotides 438-468 of the sense strand of the enterovirus and rhinovirus; and SEQ ID NO: 103 was generated to nucleotides 530-550 of the antisense strand of the enterovirus and rhino virus. SEQ ID NO: 104 and 105 are pairs for chlamydia pneumoniae (GenBank # L06108). SEQ ID NO: 104 was generated to nucleotides 265-285 of the sense strand of L06108; and SEQ ID NO: 105 was generated to nucleotides 427-447 of the antisense strand of L06108. SEQ ID NO: 106 and 107 are pairs RSV (GenBank # M74568). SEQ ID NO: 106 was generated to nucleotides 6221-6241 of the plus strand of M74568; and SEQ ID NO: 107 was generated to nucleotides 6379-6399 of the minus strand of M74568. SEQ ID NO: 108 and 109 are pairs parainfluenza 3 (GenBank # NC_{—}001796). SEQ ID NO: 108 was generated to nucleotides 7594-7614 of the plus strand of NC_{—}001796; and SEQ ID NO: 109 was generated to nucleotides 7761-7781 of the minus strand of NC_{—}001796. SEQ ID NO: 110, 111, 112, and 113 are pairs for RSV (GenBank# M74568). SEQ ID NO: 110 and 111 were used to generate a 187 basepair amplicon; SEQ ID NO: 110 was generated to nucleotides 12812-12833 of the plus strand of M74568; and SEQ ID NO: 111 was generated to nucleotides 12978-12999 of the minus strand of M74568. SEQ ID NO: 112 and 113 were used to generate a 137 nucleotide amplicon; SEQ ID NO: 112 was generated to nucleotides 13928-13948 of the plus strand of M74568; and SEQ ID NO: 113 was generated to nucleotides 14044-14065 of the minus strand of M74568.

The extracted nucleic acid molecules obtained from the sample may be amplified using a single pair of nucleotide primers directed to conserved nucleotide sequences of the pathogens of interest. However, it is preferred that two or more pairs of nucleotide primers are used in a PCR reaction. This process is referred to herein as “multiplex PCR”. Multiplex PCR permits several different genetic sequences of many different pathogens to be amplified in one process. Each of the amplified nucleic acids are labeled and then exposed to a pathogen detection region of an apparatus as disclosed herein.

In contrast to existing nucleic acid primers for viruses, the newly designed primers of the present invention (SEQ ID NO: 92-113) for respiratory viruses can sensitively amplify nucleic acids of many viral pathogens. These primers can also be integrated into a single tube mixture to amplify many different viruses as multiplex PCR format. As indicated above, the current virus list includes primers for adenoviruses, influenza A virus, influenza B virus, parainfluenza 1, parainfluenza 2, parainfluenza 3, mumps virus, respiratory syncytial virus, enteroviruses and rhinovirus. The new primer pairs (SEQ ID NO: 92-113) are able to detect more virus isolates than other PCR primers of which the inventors are currently aware. The use of these primers and the design in making the primers allows detection of all important human respiratory viruses in a simplified format. This approach can also be extended to detect all possible known human pathogens by dividing all viruses into several groups with each group set up as a multiplex PCR format to amplify several families of viruses. Thus all families of viruses can be included in a limited (5-8) PCR set up.

The nucleic acids and the substrate may be provided in a kit that permits a pathogen to be detected in a sample. The kit may include nucleic acid primers for pathogens, such as respiratory viral pathogens, which can be used to amplify nucleic acids obtained from a sample. The kit may also include the necessary equipment to obtain a sample, such as a syringe, and to process the sample to extract the nucleic acid molecules therefrom. The kit can also include appropriate tags to label the extracted nucleic acids, or the amplified nucleic acids. The kit also includes an apparatus, as herein disclosed, which comprises a substrate having a plurality of pathogen identification sites containing nucleic acid probes, which may be provided in a distinct pathogen detection region. The primers of the kit may include, and/or consist essentially of the primers having the nucleotide sequences of SEQ ID NO: 92-113. The probes of the pathogen detection region may include, or consist essentially of the probes having the nucleotide sequences of SEQ ID NO: 1-91. Due to the desirability of providing an automated convenient assay, the kit may also include a scanner and analyzer to evaluate the results of the exposure of the labeled target nucleic acid molecules to the nucleic acid probes provided in the pathogen detection region of the apparatus. The scanner and analyzer may be separate components or devices, or may be integrally provided in the kit. In addition, the scanner and analyzer may be provided as a component of the substrate to improve the automation of the assay.

### Example 1

Pathogen nucleotide sequences were obtained from the nucleotide database of NCBI (National Center for Biotechnology Information) available on the internet. The sequences for different strains or groups of pathogens were aligned using GCG software. Conserved sequences were used to design PCR primers, and the internal nucleotide sequences (e.g., the sequences flanked by the primers) of the amplified PCR products were used as nucleic acid probes on the microarray substrate.

The synthesized probes were between 66 and 75 bases long. Either a single stranded sense strand or a single stranded antisense strand oligonucleotide was used to make the microarray. The probes were chemically synthesized by Operon, Inc (Alameda, Calif.).

Pathogens spotted on the microarray included adenoviruses, influenza A virus, influenza B virus, influenza C, parainfluenza 1, parainfluenza 2, parainfluenza 3, parainfluenza 4, mumps virus, respiratory syncytial virus, enteroviruses and rhinovirus, rubella virus, coronavirus, chlamidia pneumonia and mycoplasma pneumonia. Different serotypes or subtypes of each virus family are also included. The probes that were printed on the microarray substrate are identified in Table I.

Probes were resuspended as 10 μM solution in 50% dimethyl sulfoxide (DMSO). The pathogen microarrays were printed on polylysine coated glass slide using standard printing method on Arrayer microarray machine (Genetic Microsystem) by University of California Irvine Microarray Core Facility.

Specific primers were designed based on computer sequence alignment of different pathogens, as described above. The mainly targeted pathogens that infect respiratory systems, such as rhinoviruses, adenovirus, influenza A viruses, and the like. The primers used are provided in Table II.

Total nucleic acid (DNA and RNA) were extracted from pathogen samples using a commercial kit (ZYMO RESEARCH, Orange, Calif.).

The RNA of the extracted nucleic acid was reverse transcribed to convert RNA into cDNA using AMV reverse transcriptase from Promega (Madison, Wis.). In particular, 2 μl of total nucleic acid, 2 μl 5× Reaction Buffer, 0.5 μl RNasin, 2 μM hexamer, 1.5 μl 2.5 mM dNTP, 1 μl AMV reverse transcriptase, in total 10 μl reaction. The reaction took place at 42° C. for 1 hour.

The nucleic acids were amplified using two sets of multiplex PCR primers mixture to include all the following listed respiratory pathogens. Primer mixture I contained primers for the following pathogens: adenoviruses, influenza A virus, influenza B virus, parainfluenza 1, parainfluenza 3, respiratory syncytial virus, enteroviruses and rhinovirus. Primer mixture II contained primers for the following pathogens: influenza C, rubella virus, parainfluenza 2, parainfluenza 4, mumps virus, coronavirus, chlamidia pneumonia and mycoplasma pneumonia. Multiplex PCR reactions were completed in 50 μl of total reaction volume containing: 2 μl of the above reverse transcript reaction, 2 mM MgCl₂, 0.5 μM of each primer, 200 μM of each of dCTP, dGTP and dATP; 20 μM dTTP and 100 μM of Cy3 dUTP (Amesham), 2.5 u of Taq polymerase, 50 mM KCl, 10 mM Tris-HCl, pH8.3. PCR cycles started with 95° C. for 30 seconds, then followed by 35 cycles of: 94° C., 30 seconds, 52° C. for 30 seconds, 72° C. for 120 seconds followed by another incubation at 72° C. for 7 minutes.

The PCR products were used to hybridize to the nucleic acid probes of the microarray. The hybridization mixture contained 10 μl of PCR product in 50 μl of 3×SSC buffer at 42° C. for 120 minutes. After hybridization, the slides were washed 2 times in 2×SSC, 0.1% SDS washing solution, followed by one wash of 0.1×SSC. All washes were conducted at 37° C. After the washing was completed, the slides were rinsed briefly in water and dried.

The hybridized slides were scanned on GSI Lumonics ScanArray 4000 glass slide scanner for Cy3 dye and recorded as scanning data. The intensity of individual microarray spots was quantified using computer Quantarray software to determine the relative intensity of hybridization signal.

Throughout this disclosure, a number of references including patents, patent applications, and patent publications have been referenced. All of these references are hereby incorporated by reference in their entireties.

While this invention has been described with respect to various examples and embodiments, it is to be understood that the invention is not limited thereto and that it can be practiced within the scope of the following claims.

1. Apparatus for detecting the presence of a microbe in a sample, comprising:
a substrate having a plurality of microbe identification sites, each microbe identification site having a unique address indicative of the position of that microbe identification site on the substrate; and
groups of nucleic acid probes disposed at the microbe identification sites, each group of nucleic acid probes being complementary to a target nucleic acid so as to provide a detectable signal at one or more microbe identification sites.

2. The apparatus of claim 1, wherein the microbe identification sites comprise pathogen identification sites.

3. The apparatus of claim 1, wherein the microbe identification sites comprise viral identification sites.

4. The apparatus of claim 1, wherein the microbe identification sites comprise bacterial identification sites.

5. The apparatus of claim 1, wherein the microbe identification sites comprise pathogenic and non-pathogenic identification sites.

6. The apparatus of claim 1, wherein the microbe identification sites comprise cellular and acellular pathogen identification sites.

7. The apparatus of claim 1, wherein the nucleic acid probes comprise nucleotide sequences that are complementary to genetic sequences of viruses or viroids.

8. The apparatus of claim 1, wherein the nucleic acid probes comprise nucleotide sequences that are complementary to genetic sequences of respiratory viruses.

9. The apparatus of claim 1, wherein the nucleic acid probes comprise nucleotide sequences that are complementary to a nucleotide sequence of a pathogen selected from the group consisting of: adenoviruses, influenza A virus, influenza B virus, influenza C virus, parainfluenza 1, parainfluenza 2, parainfluenza 3, parainfluenza 4, mumps virus, respiratory syncytial virus, enterovirus, rhinovirus, rubella virus, coronavirus, chlamidia pneumonia, and mycoplasma pneumonia.

10. The apparatus of claim 1, wherein the nucleic acid probes are complementary to a sequence of the target nucleic acid that has at least 80% homology among different types of microbes from a microbe family.

11. The apparatus of claim 10, wherein the nucleic acid probes are complementary to a sequence of the target nucleic acid that has at least 90% homology among different types of microbes from a microbe family.

12. The apparatus of claim 10, wherein the nucleic acid probes are complementary to a sequence of the target nucleic acid that has at least 98% homology among different types of microbes from a microbe family.

13. The apparatus of claim 1, wherein the nucleic acid probes comprise between 65 nucleotides and 80 nucleotides.

14. The apparatus of claim 13, wherein the nucleic acid probes comprise at least 70 nucleotides.

15. The apparatus of claim 13, wherein the nucleic acid probes comprise between 70 nucleotides and 75 nucleotides.

16. The apparatus of claim 1, wherein the nucleic acid probes comprise a nucleotide sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, and SEQ ID NO: 91.

17. The apparatus of claim 1, wherein a group of nucleic acid probes comprises a plurality of nucleic acids, each nucleic acid of a group having an identical nucleotide sequence.

18. The apparatus of claim 1, wherein the nucleic acid probes are printed on the substrate.

19. The apparatus of claim 1, wherein the substrate comprises a glass slide.

20. The apparatus of claim 1, wherein the substrate comprises a polylysine-coated glass slide.

21. The apparatus of claim 1, wherein the substrate comprises at least one pathogen detection region, and the microbe identification sites are located in the at least one pathogen detection region.

22. The apparatus of claim 1, wherein the target nucleic acid comprises a plurality of nucleic acids amplified by a polymerase chain reaction.

23. The apparatus of claim 1, wherein the target nucleic acid comprises a label attached thereto.

24. The apparatus of claim 23, wherein the label comprises a fluorescent label.

25. The apparatus of claim 23, wherein the label comprises Cy3.

26. A kit comprising the apparatus of claim 1, and further comprising a plurality of nucleic acid primers structured to hybridize to different regions of a target nucleic acid of a microbe to form a nucleic acid comprising about 70 to about 75 bases after a polymerase chain reaction.

27. The kit of claim 26, comprising at least one pair of nucleic acid primers structured to hybridize to a target nucleic acid of a single microbe.

28. The kit of claim 26, comprising at least two pairs of nucleic acid primers structured to hybridize to a target nucleic acid of a microbe.

29. The kit of claim 26, wherein at least one of the nucleic acid primers comprise a nucleotide sequence selected from a group consisting of: SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111,SEQ ID NO: 112, and SEQ ID NO: 113.

30. The kit of claim 26, further comprising a scanner positioned to receive signals from the apparatus and to scan the microbe detection region for a detectable signal.

31. The kit of claim 26, further comprising an analyzer in communication with the scanner to receive data from the scanner.

32. An apparatus for detecting the presence of a pathogen in a sample, comprising a nucleic acid probe disposed on a substrate, and that hybridizes to a target nucleic acid of a pathogen, the nucleic acid probe comprising a nucleotide sequence selected from a group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, and SEQ ID NO: 91.

33. The apparatus of claim 32, comprising a plurality of nucleic acid probes arranged in groups on the substrate, each group of nucleic acid probes comprising a nucleotide sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, and SEQ ID NO: 91.

34. A method for detecting the presence of a microbe in a sample, comprising:
a) identifying a target nucleic acid of a microbe in a sample;
b) labeling the target nucleic acid;
c) providing a substrate that has different groups of nucleic acid probes at different locations on the substrate; and
d) exposing the labeled target nucleic acid to the substrate such that the labeled target nucleic acid will hybridize to nucleic acid probes that have a nucleotide sequence complementary to the nucleotide sequence of the labeled target nucleic acid.

35. The method of claim 34, wherein step (a) comprises identifying a target nucleic acid of a microbe suspected of being present in a biological sample.

36. The method of claim 35, wherein the biological sample comprises a biological fluid.

37. The method of claim 36, wherein the biological fluid is selected from a group consisting of blood, serum, mucus, urine, sputum, saliva, cerebral spinal fluid, and perspiration.

38. The method of claim 34, further comprising a step of amplifying the target nucleic acid using at least one pair of nucleic acid primers and a polymerase chain reaction.

39. The method of claim 34, further comprising a step of amplifying the target nucleic acid using at least two pairs of nucleic acid primers and a polymerase chain reaction.

40. The method of claim 34, further comprising a step of amplifying the target nucleic acid and labeling the target nucleic acid during the amplification step.

41. The method of claim 34, further comprising a step of detecting the label at specific locations on the substrate where the labeled nucleic acids hybridized to the nucleic acid probes.

42. The method of claim 34, further comprising a step of detecting a fluorescent signal at specific locations on the substrate where the labeled nucleic acids hybridized to the nucleic acid probes.

43. The method of claim 34, wherein step (a) comprises identifying a target nucleic acid of a plurality of microbes suspected of being present in a sample.

44. The method of claim 34, wherein step (a) comprises identifying a target nucleic acid of a virus or viral particle.

45. The method of claim 34, wherein step (a) comprises identifying a target nucleic acid of a pathogenic or non-pathogenic bacteria.

46. The method of claim 34, wherein step (a) comprises identifying a target nucleic acid of a cellular or acellular microbe.


SEQUENCE LISTING (113 sequences)

SEQ ID NO: 1 - DNA, 73 bp, Poliovirus 2
vrsgttccgccacggacttgcgcgttacgacaagccaatcactggttcgtgaccgcttgctccgtggttagga

SEQ ID NO: 2 - DNA, 70 bp, enterovirus 70
tccgccacagacttgcgcattacgacaaaccactcactggattgtgagcatttgctccgtggttgggatt

SEQ ID NO: 3 - DNA, 70 bp, coxsackie virus A24
tccgccacggacttgcgcgttacgacaggctggctgctggattgcaactacctgctccatggttaggatt

SEQ ID NO: 4 - DNA, 70 bp, enterovirus 71
ccgctgcagagttgcccgttacgacacaccactcactggtttgtgagcatgtgctccgcagttgggatta

SEQ ID NO: 5 - DNA, 70 bp, rhinovirus 87
tccgccacggacttgcgcgttacgacaagcaacccactggtttgtgagcacttgctccatggttaggatt

SEQ ID NO: 6 - DNA, 70 bp, echovirus 30
tccgctgcagagttgcccgttacgacaggctactcactggtctgtgagcacctgctccgcagttaggatt

SEQ ID NO: 7 - DNA, 70 bp, echovirus 8
tccgctgcagagttgcccgttacgacaggccacccactggattgtgagcacctgctccgcagttaagatt

SEQ ID NO: 8 - DNA, 75 bp, Echovirus 3
chvrstccgctgcagagttacccgttacgacagcctgcccactggattgtgagtacttgctccgcagttaggatt

SEQ ID NO: 9 - DNA, 70 bp, Ev Yanbian Virus
tccgctgcagagttgcccgttacgacatgccaccctctggattgagggcacatgctccgcagttaggatt

SEQ ID NO: 10 - DNA, 70 bp, Coxsackie Virus A21
tccgccacagacttacgcattacgacaacctactcactgggtcgtgagcgattgctccgtggttaggatt

SEQ ID NO: 11 - DNA, 70 bp, echovirus 25
tccgccgcagagttacccattacgacaggttgcccactggtttgtgggtgcctgctccgagattaggatt

SEQ ID NO: 12 - DNA, 70 bp, echovirus 8
tccgctgcagagttgcccgttacgacacgccaccctctgggttgagagcacgtgctccgcagttaggatt

SEQ ID NO: 13 - DNA, 70 bp, echovirus 2
ccgctgcagagttgcccattacgacaggctgcccactggctcgtgggtacctgctccgcagttaggatta

SEQ ID NO: 14 - DNA, 70 bp, Coxsckie virus B2
tccgctgcagagttgcccgttacgacacgccatcctctggattgaggtcgcgtgctccgcagttgggatt

SEQ ID NO: 15 - DNA, 70 bp, Echovirus 11
ccgctgcagagttgcccgttacgacacactgccccttggattaggggtatgtcgtccgcagttaggatta

SEQ ID NO: 16 - DNA, 70 bp, Coxsackie Virus B5
tccgctgcagagttgcccgttacgacacgccaccccctggaatggaggcacgtgctccgcagttaggatt

SEQ ID NO: 17 - DNA, 71 bp, Echovirus 9
tccgctgcagagttacccgttacgacagactgcccactggcttgtgggtgtctgctccgcagttgggatta

SEQ ID NO: 18 - DNA, 70 bp, Coxsackie Virus A9
tccgctgcagagttgcccgttacgacacgctgccccctggtttgagggtgcgtgctccgcagttgggatt

SEQ ID NO: 19 - DNA, 70 bp, Coxsackie Virus B4
tccgctgcagagttgcccgttacgacacaccactcgctggcttgcgaacgtgtgctccgcagttaggatt

SEQ ID NO: 20 - DNA, 70 bp, Ev Stuttgart Virus
tccgctgcagagttgcccgttacgacagactactcgctggtttgctagcgtctgctccgcagttaggatt

SEQ ID NO: 21 - DNA, 70 bp, Echovirus 12
tccgctgcagagttgcccattacgacaagccacccactgggttgtgggcacttgctccacagttaggatt

SEQ ID NO: 22 - DNA, 70 bp, Coxsackie
tccgctgcagagttgcccgttacgacacactgccccctgggtcgagggtatgtgctccgcagttaggatt

SEQ ID NO: 23 - DNA, 70 bp, Coxsackie A16
tccgctgcagagttacccgttacgacacactacccactggtttgtgggcatgtgctccgcagttaggatt

SEQ ID NO: 24 - DNA, 69 bp, Echovirus 5
ccgctgcagagttacccattacgacaggctgcccactggctcgtgggtgcctgctccgcagttaggatt

SEQ ID NO: 25 - DNA, 70 bp, Porcine ev virus 9
ccgccacagagttgcccgttacgacgccctgccagctggattgctggtggacgctccgtggttaggatta

SEQ ID NO: 26 - DNA, 70 bp, Coxsackie virus b6
tccgctgcggagttgcccattacgacaagctgctcgctggtctgcgagtgcctgctccgcagttaggatt

SEQ ID NO: 27 - DNA, 69 bp, Echovirus 7
tccgctgcagagttgcccgttacgacagactgcccactggctgtgggtatctgctccgcagttaggatt

SEQ ID NO: 28 - DNA, 66 bp, Echovirus 27
ccgctgcagagttgcccgttacgacacacaccacggttgtgggcatgtgctccgcagttaggatta

SEQ ID NO: 29 - DNA, 70 bp, Echovirus 1
tccgctgcagagttacccattacgacacactgctccctggattgggagtatgtgctccgcagttaggatt

SEQ ID NO: 30 - DNA, 69 bp, Human rhinovirus
tccgtcccacagttgcccattacgaccaactacgcattggtttatgcgcattggatgtggggttggatt

SEQ ID NO: 31 - DNA, 70 bp, Ev sp virus
tccgctgcagagttgcccattacgacagaatacccgctggcgtgcgggcatctgctccgcagttgggatt

SEQ ID NO: 32 - DNA, 70 bp, Humanrhino virus
cccgtcccggaattgctcattacgaccttacaaccactggatcgtggcataaggctctagggttaaggtt

SEQ ID NO: 33 - DNA, 70 bp, Humanrhino virus
cccatcccgcaattgctcattacgaccataagctcattggtttatgagccatggctgcaggtttaaggtt

SEQ ID NO: 34 - DNA, 69 bp, Bovine ev
tccgcctccaacttacgcattacgacgtagcaacactgggttgtgcgcacacgctcggaggttgggatt

SEQ ID NO: 35 - DNA, 70 bp, Coxsackie b1
tccgctgcagagttgcccgttacgacacactgccccctggtttgagggtatgtgctccgcagttaggatt

SEQ ID NO: 36 - DNA, 70 bp, Poliovirus I
aagctaaaaggcacagagagcgaacgtgatcctgagtgttcctaggatctttagtccatctaattgattc

SEQ ID NO: 37 - DNA, 70 bp, Poliovirus 3
agccaatagacacaaggagcgtacatggtcctgcgtattccgaggatcttttgtccatctgattgattca

SEQ ID NO: 38 - DNA, 70 bp, Enterovirus 70
aagtttcttcaccattgtgccaggcagtaggcacaaggatctcacatgatcttgtgtgtttctcggatct

SEQ ID NO: 39 - DNA, 70 bp, Poliovirus 1
ctccgaattaaagaaaaatttacccctacaacagtatgacccaatccaattcgactgaggtagggttact

SEQ ID NO: 40 - DNA, 70 bp, Human rhinovirus
ataaactcctacttctactcaaattaagtgtctatattgttaacctaaaagaggtccaaccagcgcctcc

SEQ ID NO: 41 - DNA, 70 bp, Human beta-actin
cagcactgtgttggcgtacaggtctttgcggatgtccacgtcacacttcatgatggagttgaaggtagtt

SEQ ID NO: 42 - DNA, 70 bp, Human GAPDH
atccacagtcttctgggtggcagtgatggcatggactgtggtcatgagtccttccacgataccaaagttg

SEQ ID NO: 43 - DNA, 70 bp, h. cycphilin
gatggacttgccactagtgccattatggcgtgtgaagtcaccaccctgacacataaaccctggaataatt

SEQ ID NO: 44 - DNA, 70 bp, Human P 53 virus
gcctctggcattctgggagcttcatctggacctgggtcttcagtgaaccattgttcaatatcgtccgggg

SEQ ID NO: 45 - DNA, 70 bp, Human Sod1 virus
gcaggccttcagtcagtcctttaatgcttccccacaccttcactggtccattactttccttctgctcgaa

SEQ ID NO: 46 - DNA, 70 bp, Human Nos1 virus
gaggaccacgtgggtctcagaggcaatgcctctgagtacctccagggcgctgtcatagctcaggtccacc

SEQ ID NO: 47 - DNA, 74 bp, adenovirus
ggccgaaatcgcctatcaagacaactcaggagacgtgcaggagattttgcgccaggccgccgtcaacgacaccg

SEQ ID NO: 48 - DNA, 74 bp, adenovirus
tccaacgacctcgccgccaccgtggagcgagccggacgcggagatctccaggaggaagagatcgagcagttcat

SEQ ID NO: 49 - DNA, 74 bp, adenovirus
ccgagattgcctaccaggacaactcgggcgacgtgcaagagattctgcggcaggccgccgtcaatgataccgag

SEQ ID NO: 50 - DNA, 74 bp, adenovirus
cgagatcgcctaccaggacaactcgggcgacgtgcaagagatcctgcgtcagcccgccgtcaatgacgccgaga

SEQ ID NO: 51 - DNA, 73 bp, adenovirus
cgagatcgcctaccaggacaactctggcgacgtgcaagaaatccttaggcaagccgccgtcaacgataccgag

SEQ ID NO: 52 - DNA, 74 bp, adenovirus
ggagattgcctaccaagacaactctggcgacgtgcaggagattctgcgacaagccgcggtcaacgatgccgata

SEQ ID NO: 53 - DNA, 74 bp, adenovirus
agaaattgcctaccaagataactcaggcgacgttcaggagattttaagacaggctgcagtaaatgacactgaga

SEQ ID NO: 54 - DNA, 74 bp, measles
gtgtgcagccaaaatgccttgtacccgatgagtcctctgctccaagaatgcctccgggggtccaccaagtcctg

SEQ ID NO: 55 - DNA, 74 bp, measles
tgctcgtacactcgtatccgggtcttttgggaaccggttcattttatcacaagggaacctaatagccaattgtg

SEQ ID NO: 56 - DNA, 74 bp, Parainfluenza virus 1
tatatgcgtattcatcaaacttaatcactcaaggatgtgcagatatagggaagtcatatcaggttttacaatta

SEQ ID NO: 57 - DNA, 74 bp, Parainfluenza virus 3
  misc_feature (48)..(48): n at position 48 is g
ggtaacaagatctatatatatacaagatctacaagttggcatagcaanttacaattaggaataattgatattac

SEQ ID NO: 58 - DNA, 74 bp, Parinfluzenza virus 4B
cgaccgatttaaatcaatataatcaattactcaagagtgctgagaaccacatccaacggtcaaatgattactta

SEQ ID NO: 59 - DNA, 74 bp, Parainfluenza virus 4A
caactgatttaaatcaatacaatcaattactcaagagtgctgaagatcacatccaacgatcaactgattactta

SEQ ID NO: 60 - DNA, 74 bp, Parainfluneza virus 2
tagaacagaggaaagaagagttgcatcaatggcatatattaaaggtgccacacacagtttgaaggctgctctta

SEQ ID NO: 61 - DNA, 74 bp, Mumps virus
ccaaaacagatgaacgaagggttgcatcaatggcttatatcaaaggggcatcagtatcacttaaatcagcactc

SEQ ID NO: 62 - DNA, 74 bp, Mumps virus
  misc_feature (14)..(14): n at position 14 is a g or a
ctaaaacagatgancggagggttgcgtcaatggcttacatcaaaggagcatctgtatcacttaaatcagcactc

SEQ ID NO: 63 - DNA, 74 bp, Respitory syncytial virus
cacatctctaggagccattgtgtcatgctatggcaaaactaaatgtacagcatccaataaaaatcgtggaatca

SEQ ID NO: 64 - DNA, 74 bp, respiratory syncytial virus
tacttctcttggagctatagtgtcatgctatggtaaaactaaatgcactgcatccaacaaaaatcgtgggatta

SEQ ID NO: 65 - DNA, 74 bp, Influenza A
cagagacaagaagatgtctttgcagggaaaaacactgatcttgaggctctcatggaatggctaaagacaaaacc

SEQ ID NO: 66 - DNA, 74 bp, Influenza A
  misc_feature (36)..(36): n at position 36 is c or a
cagagacttgaagatgtctttgctgggaaaaacacngatcttgaggctctcatggaatggctaaagacaagacc

SEQ ID NO: 67 - DNA, 74 bp, Influenza A
  misc_feature (36)..(36): n at position 36 is c or a
cagagacttgaagatgtctttgctgggaagaacacngatcttgaggctctcatggagtggctaaagacaagacc

SEQ ID NO: 68 - DNA, 74 bp, Influenza A
  misc_feature (36)..(36): n at position 36 is c or a
cagagacttgaagatgtctttgcagggaaaaacacngaccttgaagcactcatggaatggctaaagacaagacc

SEQ ID NO: 69 - DNA, 74 bp, Influenza A
cagagacttgaagatgtttttgctggaaagaatactgacctcgaggctctcatggaatggctaaagacaagacc

SEQ ID NO: 70 - DNA, 74 bp, Influenza B
caggaatgggaacaacagcaacaaaaaagaaaggcctgattctagctgagagaaaaatgagaagatgtgtgagc

SEQ ID NO: 71 - DNA, 74 bp, Influenza B
caggaatgggaacaacagcaacaaagaagaaaggcctaattctagctgagagaaaaatgagaagatgtgtaagc

SEQ ID NO: 72 - DNA, 74 bp, Influenza C
  misc_feature (28)..(47): n at position 28 is c , n at position 47 is c
agcctgcaaatcagcagctaaactgatnaagaatgaacatcttcccntaatgtctggagaagccaccacaatgc

SEQ ID NO: 73 - DNA, 74 bp, rhinovirus
aaccttaaacctgcagccagtgcacacaatccagtgtgtagctggtcgtaatgagcaattgcgggatgggacca

SEQ ID NO: 74 - DNA, 73 bp, rhinovirus
aaccttaaccctgcagctagagcgcgcaaaccagcgtgtttctagtcgtaatgagcaattgcgggatgggacc

SEQ ID NO: 75 - DNA, 73 bp, rhinovirus
aaccttaaccctgcagctagtgcatgcaatccagcatgttgctagtcgtaatgagcaattgcgggatgggacc

SEQ ID NO: 76 - DNA, 73 bp, rhinovirus
aaccttaaccctgcagctagtgcatgtaatccaacatgttgctagtcgtaatgagcaattgcgggacgggacc

SEQ ID NO: 77 - DNA, 74 bp, rhinovirus
aaccttaaacctgcagccatggctcataaaccaatgagcttatggtcgtaatgagcaattgcgggatgggaccg

SEQ ID NO: 78 - DNA, 75 bp, rhinovirus
aaccttaaccccgcagccgctgcccatggatccagtgggtatacggtcgtaacgcgcaatgtggggatgggacca

SEQ ID NO: 79 - DNA, 74 bp, rhinovirus
aaccttaaacccgcagccaaggtgtgcaagccagcatattcttggtcgtaacgagcaattgtgggatgggacca

SEQ ID NO: 80 - DNA, 74 bp, rhinovirus
aatcctaaccatggagcaagtgctcacaaaccagtgggttgcttgtcgtaacgcgcaagtccgtggcggaaccg

SEQ ID NO: 81 - DNA, 74 bp, rhinovirus
aaccttaaacccgcagccatggttcataaaccaatgagcttatggtcgtaatgagcaattgtgggatgggaccg

SEQ ID NO: 82 - DNA, 73 bp, Coronavirus
caaaggagttgttggtgttttgaccttagacaaccaagatcttaatgggaatttctatgacttcggtgacttt

SEQ ID NO: 83 - DNA, 74 bp, Rubella
ccaccgacaccgtgatgagcgtgttcgcccttgctagctacgtccagcaccctcacaagaccgtccgggtcaag

SEQ ID NO: 84 - DNA, 74 bp, Rubella
ccaccgacaccgtgatgagtgtgttcgccctcgccagctacgtccagcacccccacaagaccgtcagggtcaag

SEQ ID NO: 85 - DNA, 74 bp, Mycoplasma pneumoniae
aatgggactgagacacggcccatactcctacgggaggcagcagtagggaatttttcacaatgagcgaaagcttg

SEQ ID NO: 86 - DNA, 74 bp, Chlamydia pneumoniae
cgtctaggcggattgagagattgaccgccaacactgggactgagacactgcccagactcctacgggaggctgca

SEQ ID NO: 87 - DNA, 74 bp, parvovirus
ttgggtatactttccccctcaatatgcttacttaacagtaggagatgttaacacacaaggaatctctggagaca

SEQ ID NO: 88 - DNA, 22 bp, adenovirus
  misc_feature (10)..(16): n at position 10 is c , n at position 16 is c
ccctgaaagavagttcvacaga

SEQ ID NO: 89 - DNA, 22 bp, adenovirus
  misc_feature (2)..(14): n at position 2 is a or c or t, n at position 11 is c or a, n at position 14 is  c or g
tntacagccgngtntggaacga

SEQ ID NO: 90 - DNA, 21 bp, parainfluenza 1
accttcattatcaattggtga

SEQ ID NO: 91 - DNA, 22 bp, parainfluenza 1
tcctgttgtcgttgatgtcata

SEQ ID NO: 92 - DNA, 20 bp, mump virus
  misc_feature (15)..(18): n at position 15 is a or g or t, n at position 18 is a or t
ccacccatcagagtnccnta

SEQ ID NO: 93 - DNA, 20 bp, parainfluenza virus
  misc_feature (6)..(12): n at position 6 is a or t, n at position 12 is a or t
ccgaangcccanatatatac

SEQ ID NO: 94 - DNA, 23 bp, influenza A virus
  misc_feature (3)..(3): n at position 3 is g or a
ccntcaggccccctcaaagccga

SEQ ID NO: 95 - DNA, 21 bp, influenza A virus
  misc_feature (4)..(13): n at position 4 is a or g or t, n at position 13 is a or c or t
aaancgtctacgntgcagtcc

SEQ ID NO: 96 - DNA, 24 bp, inflenza B virus
gaatggataaaaaacaaaagatgc

SEQ ID NO: 97 - DNA, 23 bp, infleuenza B virus
tggccttctgctatttcaaatgc

SEQ ID NO: 98 - DNA, 21 bp, entrovirus
  misc_feature (19)..(19): n at position 19 is c or t
tcctccggcccctgaatgngg

SEQ ID NO: 99 - DNA, 23 bp, rhinovirus
  misc_feature (6)..(20): n at position 6 is a or t, n at position 20 is c or t
cacggncacccaaagtagtnggt

SEQ ID NO: 100 - DNA, 21 bp, chylamydia pneumoniae
ggtaaaagcccaccaaggcga

SEQ ID NO: 101 - DNA, 22 bp, chylamydia pneumoniae
gaaagtgctttacaaccctaag

SEQ ID NO: 102 - DNA, 21 bp, respiratory syncytial virus
  misc_feature (1)..(1): n at position 1 is c or t
nttaaccagcaaagtgttaga

SEQ ID NO: 103 - DNA, 19 bp, respiratory syncytial virus
  misc_feature (7)..(7): n at position 7 is a or t
aggtgtngttacacctgca

SEQ ID NO: 104 - DNA, 22 bp, parainfluenza 3 virus
cagatgtatatcaactgtgttc

SEQ ID NO: 105 - DNA, 21 bp, parainfluenza 3 virus
tccctggtccaacagatgggt

SEQ ID NO: 106 - DNA, 22 bp, respiratory syncytial virus
gtgatacaaaaacagcatatgt

SEQ ID NO: 107 - DNA, 21 bp, respiratory syncytial virus
tccaatgtccagctaaattag

SEQ ID NO: 108 - DNA, 21 bp, respiratory syncytial virus
tgcatgcttccttggcatcat

SEQ ID NO: 109 - DNA, 21 bp, respiratory syncytial virus
gctccttcacctatgaatgct

SEQ ID NO: 110 - DNA, 70 bp, Poliovirus 1
tccgccacggacttgcgcgttacgacaggccaatcactggtttgtgaccacctgctccgaggttgggatt

SEQ ID NO: 111 - DNA, 70 bp, Echo virus 4
tccgctgcggagttacccattacgacacaccactcactggcttgtgagcgtgtgctccgcagttaggatt

SEQ ID NO: 112 - DNA, 70 bp, Coxsackie virus B6
tccgccgcagagttgcccgttacgacagactgcccactggtgtgtgggtgtctgctccgcggttaggatt

SEQ ID NO: 113 - DNA, 70 bp, Poliovirus 3
tccgccacagactttcgcgttacgacaggcaaaccactggtttgtgaccacctgctccgtggttgggatt